(19) United States
(12) Patent Application Publication
(10) Pub. No.: US 2008/0166753 A1
(43) Pub. Date: Jul. 10, 2008

(54) Microbial Growth Assay

(75) Inventors: Douglas Storey, Calgary (CA); Tyler Field, Belfast (IE); Gordon Ramage, Scotland (GB)

Correspondence Address: FISH & RICHARDSON P.C., PO BOX 1022, MINNEAPOLIS, MN, 55440-1022, US

(73) Assignee: University Technologies International Inc., Calgary (CA)

(21) Appl. No.: 10/593,951

(22) PCT Filed: Mar. 2, 2005
(86) PCT No.: PCT/CA2005/000312

§ 371 Date: Oct. 18, 2007

Related U.S. Application Data
(60) Provisional application No. 60/561,766, filed on Apr. 12, 2004.

(51) Int. Cl. C12Q 1/18; C12M 1/00; C12M 1/34

(52) U.S. Cl. 435/32; 435/289.1; 435/305.3; 435/288.7

(57) ABSTRACT

Susceptibility of sessile microorganisms to antimicrobial agents is tested with a method comprising growing the microorganisms on a support to form a biofilm, contacting the biofilm with a metabolic substrate having a chromogenic and/or fluorogenic moiety to continue a base-line metabolic activity of the biofilm, contacting the biofilm with one or more antimicrobial agents, determining an experimental metabolic activity of the biofilm by measuring a signal from the metabolic substrate, and comparing the base-line metabolic activity with the experimental activity. The biofilm may be grown in a device comprising a plurality of wells, each well having a planar bottom and at least on wall, a plurality of supports comprising discs for growing the biofilm, each support disposed within a well perpendicular to the planar bottom, and at least one cover that fittably seals the top of each well.

### TECHNICAL FIELD

This invention relates to methods and devices for the analysis of microbial biofilms, and sensitivity of such biofilms to anti-microbial agents (antibiotics, disinfectants and biocides).

### BACKGROUND

The use of medical devices has facilitated patient care and improved the treatment of diseases and disorders. However, with improved clinical management, there has been a significant drawback. The introduction of artificial materials into numerous anatomical sites has been accompanied by the ability of microbes to colonize and form biofilms (Costerton et al., Science 284(5418):1318-22, 1999; Donlan, Clin Infect Dis. 33(8):1387-92, 2001; Donlan, Emerg. Infect. Dis. 7(2):277-81, 2001; Khardori et al. J. Ind. Microbiol. 15(3):141-7, 1991). These medical devices provide a sanctuary for microbes from the hostile surrounding environment. Accordingly, the ability to monitor growth and susceptibility of such microbes is important in reducing infection, morbidity and mortality.

### SUMMARY

Disclosed are methods and devices for susceptibility testing of sessile organisms. In this era of widespread increased antimicrobial resistance and increased use of indwelling devices it is crucial to establish methodologies that allow evaluation of current and new antimicrobial agents against cells in biofilm. This painstaking work has been previously developed for many planktonic organisms; however, the consideration of a sessile microbial lifestyle appears to have been so far neglected. The increased resistance phenotype of sessile organisms emphasizes the need for a standardized assay to test biofilm antimicrobial susceptibilities.

The devices and methods provided by the disclosure allow for an efficient and automated biofilm killing assay that has particular use with 96 well platforms commonly used in many diagnostic assay systems.

Provided by the disclosure is a rapid, inexpensive, easy to use, accurate and reproducible methodology for biofilm susceptibility testing, that benefits from the use of a calorimetric method to assess the effects of both antibiotics and disinfectants against biofilm cells.

The disclosure provides a method of determining the susceptibility of a biofilm to an antimicrobial agent. The method comprises culturing microbes on a support to form a biofilm; contacting the biofilm with a metabolic substrate; determining a base-line metabolic activity of the biofilm by measuring a signal from the metabolic substrate; contacting the biofilm with one or more antimicrobial agents; determining an experimental metabolic activity by measuring a signal from the metabolic substrate; and comparing the baseline metabolic activity with the experimental metabolic activity, wherein a change is indicative of an antimicrobial agent that affects microbes in the biofilm.

The disclosure also provides an assay device comprising a cell culture device comprising a plurality of wells, each well comprising a substantially planar bottom and at least one wall; a plurality of supports, each support disposed within a well perpendicular to the substantially planar bottom, wherein the plurality of supports comprise discs; and at least one cover that fittably seals the top of each well.

The disclosure provides an assay system comprising a cell culture device having a plurality of wells and/or channels, each well or channel comprising a substantially planar bottom; a plurality of supports, each support disposed within a well or channel perpendicular to the substantially planar bottom; and at least one cover that fittably seals the top of each well or channel; culturing a sample comprising a microbial population in a media within the wells or channels such that the media is in contact with the supports thereby forming a biofilm on the supports; measuring a fluorometric or calorimetric absorbance from a fluorogenic or chromogenic moiety in the sample; comparing the fluorogenic or calorimetric absorbance to a standard sample. In one aspect, the assay system further comprising means for measuring a base-line metabolic activity of the biofilm; means for contacting the biofilm with one or more antimicrobial agents; means for measuring an experimental metabolic activity; and means for comparing the base-line metabolic activity with the experimental metabolic activity, wherein a change is indicative of an antimicrobial agent that affects microbes in the biofilm.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-C show a view of biofilm adherent sites on a lid of a vessel; 1A shows a lid with a plurality of substantially identical projections; lB and 1C show an embodiment wherein the projections comprise a cleft for inserting and removing disposable discs or other supports;

FIG. 2 is a top view of a vessel for receiving the plural biofilm adherent sites of FIG. 1;

FIG. 3 is a side view, partly broken away, of the lid and vessel of FIGS. 1 and 2;

FIG. 4 is a side view schematic of a lid and vessel combination as shown in FIG. 3 on a tilt table; and

FIGS. 5A and B shows a top view (5A) of a 96 well plate for use with the invention; 5B shows a side view of discs disposed within the wells of the 96 well plate;

FIG. 6 shows a growth curve of an XTT assay;

FIG. 7 is a graph depicting the concentration and optimal change in absorbance;

FIG. 8 shows the growth curve of biofilms;

FIGS. 9A and B show graphs of metabolic activity of various biofilms measured by XTT absorbance assays;

FIG. 10 is a graph showing a dose-dependent effect of antibiotics on biofilms when measured by XTT assay; and

FIGS. 11A and B are graphs comparing the growth curves of biofilms measured by XTT absorbance and cfu/ml.

Like reference symbols in the various drawings indicate like elements.

### DETAILED DESCRIPTION

Antimicrobial susceptibility testing is performed routinely within hospital diagnostic laboratories. The results of these tests are essential in predicting the most effective course of antimicrobial therapy. High throughput testing in a rapid manner is the most effective way to increase clinical outcome, as well as decreasing the burden economically. The devices and methods provided herein are useful to determine the antimicrobial susceptibility (e.g., susceptible, moderately susceptible, intermediate resistant, or resistant) of one or more antimicrobial agents with respect to pathogens present in biofilms from any number of sample types.

The present disclosure involves a microbiological method, compositions and devices for the direct detection and categorical interpretation of antimicrobial susceptibility in relation to the majority of microbes in samples, such as environmental samples and biofilm samples.

Devices such as shunts, prostheses (voice, heart valves, artificial joints and the like), stents, implants (lens, breast, denture, and the like) endotracheal tubes, pacemakers, and various types of catheters have all been shown to support colonization and biofilm formation, which may be clinically problematic and adversely affect the function of the implanted device. Recently, there has been a greater awareness of the role that adherent microbial populations play in human medicine. It has been estimated that about 65% of all human microbial infections involve biofilms (Costerton et al., Science 284(5418):1318-22, 1999; Donlan, Clin Infect Dis. 33(8):1387-92, 2001; Donlan, Emerg. Infect. Dis. 7(2):277-81, 2001; Khardori et al. Donlan, Emerg. Infect. Dis. 8(9):881-90, 2002; Donlan et al., Clin. Microbiol. Rev. 15(2):167-93, 2002; Khardori et al., J. Ind. Microbiol 15(3):141-7, 1995). These adherent heterogeneous microbial populations, biofilms, have come under intense scrutiny because they are able to readily impede host immunity, and more alarmingly, resist antimicrobial therapy.

Biofilm infections, while on the increase, are seldom taken into account when susceptibility testing is performed. Sessile cells from biofilms are phenotypically distinct from their planktonic counterparts and are associated with an increased resistance phenotype. Thus, for suspected biofilm-related infections standardized testing does not provide an accurate in vitro-in vivo correlation. As a result an alternative testing strategy is needed.

Decreased susceptibility of sessile cells to antimicrobial agents when compared to planktonic cells has been reported over the past decade (Evans, et al., J. Antimicrob. Chemother., 25(4):585-91, 1991; Hoyle and Costerton, Prog. Drug Res., 37(9):91-105, 1991; Gander, J. Antimicrob. Chemother. 37(6):1047-50, 1996; Amorena, J. Antimicrob. Chemother., 44(1):43-55, 1999). However, the comparatively new field of biofilm research has progressed at such rate, that the development of assays to measure sessile antimicrobial data, often ingenious, has resulted in a plethora of different antimicrobial testing strategies. Moreover, biofilms can be quantified using a variety of techniques, such as direct microscopic enumeration, total viable plate counts, radiochemistry and luminometry. Consequently, there are a myriad of potential techniques to measure biofilm antimicrobial susceptibilities. It is, therefore, imperative that a standardized antimicrobial susceptibility testing protocol for biofilms be implemented, from a clinical, research and industrial standpoint.

Classically, microbes have been studied as single species, based on the pure culture mode of growth. Microorganisms have historically been diluted to a single cell and artificially studied in liquid culture, a strategy that has overwhelmingly predominated in the study of microbial physiology and pathogenesis in the laboratory. This is also true for susceptibility testing. Conventional methods of killing bacteria (such as antibiotics and disinfection) are often ineffective with biofilm bacteria. The huge doses of antimicrobials required to rid systems of biofilm microbes are undesirable environmentally (and perhaps not allowed by environmental regulations) and impractical medically (since what it would take to kill the biofilm microbes would also kill the patient). So new strategies based on a better understanding of how bacteria attach, grow and detach are urgently needed by many industries. In the past, treatment of biofilms has been based on empirical data obtained from planktonic susceptibility testing. Such data when transferred to the clinic has often proven to be ineffective.

A biofilm is a population of microbes that grow on devices (e.g., biomedical devices) and surfaces of a compromised host. Typical biofilms are resistant to antibiotics because of their structural composition and multiple complex mechanisms. Biofilm forms when microbes adhere to surfaces in aqueous environments and begin to excrete a slimy, glue-like substance that can anchor the microbes to all kinds of material—such as metals, plastics, soil particles, medical implant materials, and tissue. A biofilm can be formed by a single bacterial species, but more often biofilms consist of many species of bacteria, as well as fungi, algae, protozoa, debris and corrosion products. Essentially, biofilm may form on any surface exposed to bacteria and some amount of fluid. Once anchored to a surface, biofilm microorganisms carry out a variety of detrimental or beneficial reactions (by human standards), depending on the surrounding environmental conditions. Certain bacteria can attach to a surface and differentiate to form a complex, multicellular structure comprising microbial cells (e.g., algal, fungal, bacterial, and combinations thereof) and the extracellular biopolymer these cells produce. Bacteria attach to surfaces by proteinaceous appendages referred to as fimbriae. Once a number of fimbriae have “glued” the cell to the surface, the detachment of the organism becomes very difficult. Once attached, the organisms begin to produce material (an extracellular biopolymer referred to as “slime”). The slime consists primarily of polysaccarides and water. The amount of biopolymer produced can exceed the mass of the bacterial cell by a factor of 100 or more. The biofilm structure provides a favorable protective environment for the survival of the cells of the organism. A microbial organism includes any organism capable of being present in a biological sample. Such organisms include but are not limited to bacteria and fungi.

Bacteria that can form biofilms include gram-positive cocci such as, for example, Staphylococcus aureus, Streptococcus pyogenes (group A), Streptococcus sp. (viridans group), Streptococcus agalactiae (group B), S. bovis, Streptococcus (anaerobic species), Streptococcus pneumoniae, and Enterococcus sp.; Gram-negative cocci such as, for example, Neisseria gonorrhoeae, Neisseria meningitidis, and Branhamella catarrhalis; Gram-positive bacilli such as Bacillus anthracis, Bacillus subtilis, Corynebacterium diphtheriae and Corynebacterium species which are diptheroids (aerobic and anerobic), Listeria monocytogenes, Clostridium tetani, Clostridium difficile, Gram-negative bacilli such as, for example, Escherichia coli, Enterobacter species, Proteus mirablis and other sp., Pseudomonas aeruginosa, Klebsiella pneumoniae, Salmonella, Shigella, Serratia, and Campylobacter jejuni. Infection with one or more of these bacteria can result in diseases such as bacteremia, pneumonia, meningitis, osteomyelitis, endocarditis, sinusitis, arthritis, urinary tract infections, tetanus, gangrene, colitis, acute gastroenteritis, bronchitis, and a variety of abscesses, nosocomial infections, and opportunistic infections. Fungal organisms include dermatophytes (e.g., Microsporum canis and other M. sp.; and Trichophyton sp. such as T. rubrum, and T. mentagrophytes), yeasts (e.g., Candida albicans, C. tropicalis, or other Candida species), Saccharomyces cerevisiae, Torulopsis glabrata, Epidermophyton floccosum, Malassezia furfur (Pityropsporon orbiculare, or P. ovale), Cryptococcus neoformans, Aspergillus fumigatus, Aspergillus nidulans, and other Aspergillus sp., Zygomycetes (e.g., Rhizopus, Mucor), Paracoccidioides brasiliensis, Blastomyces dermatitides, Histoplasma capsulatum, Coccidioides immitis, and Sporothrix schenckii. Thus, methods for assaying the susceptibility of biofilms comprising any one or more of the microbes identified herein to antimicrobial agents can identify effective methods of treatment and/or prophylaxis.

The methods and device described here for analyzing biofilms and biofilm susceptibility are fast, efficient, reliable and reproducible, with high throughput potential. For semi-quantitative analysis of a preformed biofilm exposed to antimicrobial drugs, a fluorometric and/or calorimetric assay is provided. In one aspect, biofilms are assayed for metabolic activity using a metabolic substrate such as a tetrazolium salt which is reduced to a colored formazan by electrons emitted as a by-product of bacterial metabolism. This technology has been used previously to determine viability of homogeneous bacterial cultures (Roslev and King, Appl. Environ. Microbiol., 59(9):2891-2896, 1993) in the presence of antibiotics (Seligy and Rancourt, J Ind. Microbial. Biotechnol., 22(6):565-574, 1999; De Logu et al., Eur. J. Clin. Microbiol. Infect. Dis., 20(1):33-9, 2001) and immune system components (Stevens and Olsen, J. Immunol. Methods, 157(1-2):225-31 1993; Lin et al., Clin. Diagn. Lab. Immunol., 8(3):528-33, 2001). More recently, this technology has been adapted for use with Candida albicans as a rapid assay of antifungal susceptibility (Ramage et al., Antimicrob. Agents Chemother., 45(9):2475-9, 2001).

The assay techniques provided herein comprise the culturing of biofilms on a support (e.g., acetate discs) in multiwell culture dishes. Other useful support materials can comprise polymethylmethacrylate, glass, metal, and plastic. For example, glass coverslips; plastic THERMANOX coverslips; glass beads; multiwell dishes of plastic or glass; plastic, metal, glass, or wood discs; organic supports in the form of discs, pins or paddles; discs or paddles coated with organic material; plastic, metal, glass, or wood pins or paddles; and plastic, metal, glass, wood or organic beads. The metabolic activity of the biofilms is measured using a metabolic substrate comprising a detectable moiety such as a chromogenic and/or fluorogenic moiety. Metabolic substrates comprising chromogenic and/or fluorogenic moieties include molecules that can be metabolized by an enzyme or a group of enzymes of the microorganisms whose presence or growth ability are sought to be detected. Such substrates include, but are not limited to, hydrolyzable enzyme substrates and redox dyes. The enzymatic reaction typically involves hydrolyzing one or more covalent bonds of the substrate or transferring the reducing equivalents from a specific substrate to an acceptor. The substrates typically contain detectable moieties or can be converted to a detectable compound. Upon being metabolized by one or more microbial enzymes, the substrate generates a detectable moiety in the medium. In one aspect, the signal generating substrate is selected from the chromogenic or fluorogenic substrates of phosphatase, aminopeptidases (e.g., L-alanine aminopeptidase or L-leucine aminopeptidase), glycosidases, esterases, and sulfatases, as well as from the chromogenic or fluorogenic tetrazolium compounds (such as, e.g., sodium 3′-{1-[(phenylamino)-carbonyl]-3, 2,3-bis[2-methyloxy-4-nitro-5-sulfophenyl]-2H-tetrazolium-5-carboxanilide (XTT), 2-(p-iodophenyl)-3-(p-nitrophenyl)-5-phenyltetrazolium chloride (INT), 5-cyano-2,3-ditolyl tetrazolium chloride (CTC), 2,3,5-triphenyltetrazolium chloride (TTC), and resazurin, and the like. This list is not meant to exclude signal generating substrates which have yet to be discovered but may later be identified and included in this list by those of ordinary skill in the art. For example, a metabolic dye such as XTT can be used in the assays of the disclosure. The assay includes culturing a biofilm in the presence and absence of various antimicrobial agents and measuring metabolic activity with, for example, XTT, wherein the XTT when metabolized provides an indication of the presence of a biofilm as well as the effect an antimicrobial agent has on cell growth and viability due to a reduction in XTT metabolic product. A chromogenic and/or fluorogenic substrate does not cause or produce a detectable signal when it is affiliated with (e.g., covalently bonded to) a cleavable moiety or before the moiety is reduced and metabolized by the organisms. However, when an enzyme or a group of enzymes from viable target microbe metabolize the substrate, a chromogenic and/or fluorogenic moiety is released or formed and causes or is capable of producing a detectable signal in the medium. In one embodiment, the detectable moieties are fluorogens that produce and emit fluorescence when properly excited by an external energy source, or chromagens that produce a color change observable in the visible wavelength range (alternatively in the ultraviolet or infrared spectra). Examples of fluorogenic and/or chromogenic moieties include, but are not limited to: 4-methylumbelliferone, orthonitrophenyl, para-nitrophenyl, para-nitroanilide, 4-methoxy-J-naphthylamide, 7-amido-4-chloro-3-indoxyl, and formazan, and the like.

Detectable changes include a characteristic change in a medium or sample that is observable or measurable by physical, chemical or biological means known to those skilled in the art. Such a detectable signal may be assayed by chemical, visual, tactile, or olfactory means. For example, a change in emission or absorbency of: visible or invisible light or radio waves at a certain wavelength, electrical conductivity, emission of gas, turbidity or odor. A detectable signal may also be a change in physical state such as between solid, liquid and gas. The detectable signal may produce a chemical change, such as change in pH, which is measurable. Typically, a detectable signal is measured visually such as by a change in fluorescent or color emission of the medium.

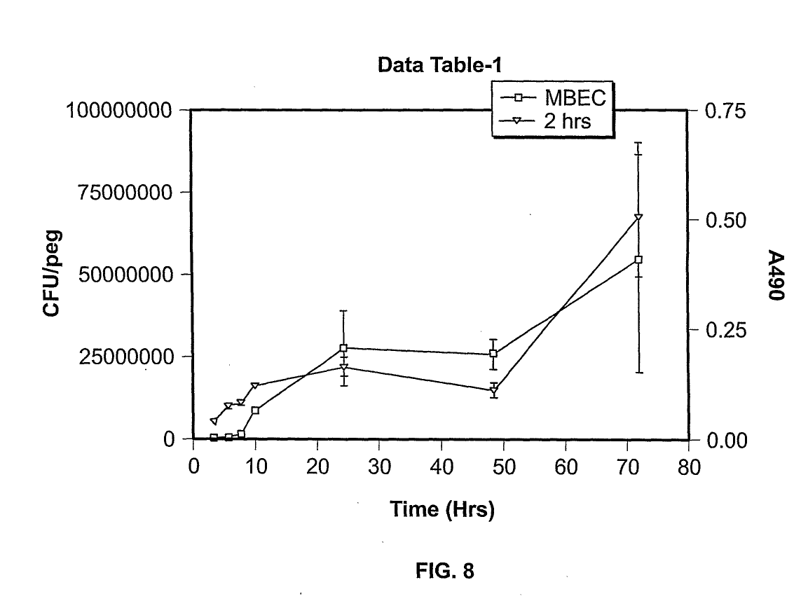

Semi-quantitative calorimetric techniques are preferred to classical total viable cell counts primarily because of the inherent problems associated with enumerating bacteria by this methodology, i.e. results are highly variable due operator handling and contamination. Colorimetric evaluation shows no bias in this respect. For example, a colorimetric XTT metabolic assay was shown to produce color changes that upon spectrophotometric determination of absorbance, exhibited no statistically significant differences between independent biofilms formed on pegs of a Calgary Biofilm Device (CBD). Demonstrated herein is that the growth curve of an XTT assay reading were proportional to cellular density of the biofilm (FIG. 6). Because of its water-solubility, the XTT-reduction assay can be easily quantified without performing additional steps such as centrifugation, addition of lysis buffer, solubilization, removal of medium and sonication. The use of such easily diffusable calorimetric substrates takes advantage of a biofilm's highly hydrated structure that allows diffusion of secondary metabolites and nutrients. Because sessile cells that resist the actions of antibiotics would continue to be metabolically active, these cells would continue to initiate color changes of, e.g., XTT, as it diffuses into the biofilm, whereas dead cells would not. Overall, using the methodology disclosed herein allows multiple parameters to be easily investigated.

In another aspect, a disc model is used. In a particular embodiment, acetate discs are used as a growth support but any material which provides a support upon which a biofilm can form may be used. Suitable disc or bead materials include plastic, metal, glass, wood or organics. Discs need not be round, but may be any shape which allows for biofilm formation. The discs serve as suitable support for biofilm growth. The discs, in particular acetate discs, are suitably inexpensive supports, which can be used to form multiple biofilms using a 96-well platform. The disc models are in contrast to other proposed techniques for the examination of antibiotic susceptibilities of biofilm cells. For example, Domingue et al. (J. Clin. Microbiol., 32(10):2564-8, 1994) proposed the use of the Modified Robbin's Device (MRD) technology to produce multiple biofilms for antimicrobial testing. While this technique is a well-recognized model, it requires expert handling, relatively few equivalent biofilms can be produced, requires longer processing times and is more open to contamination than the method and device presented herein. Formation of biofilms using other technologies such as the perfused biofilm fermenter models or membrane-associated biofilm models (Gander and Gilbert, J. Antimicrob. Chemother., 40(3):329-34, 1997; Baillie and Douglas, Antimicrob Agents Chemother., 42(8):1900-5, 1998) are not amenable to high throughput screening and require the use of specialized equipment not generally available in a clinical laboratory. The present disclosure provides methods and devices that minimize sample handling, are rapid, reproducible and allow the testing of multiple factors within a single trial (different antimicrobials, biofilm ages, growth media, and the like). One over-riding advantage of the methods and devices associate with this assay is the fact that the assay is non-destructive and does not require subsequent culture of cells following antimicrobial challenge. The calorimetric assay component of the methods disclosed herein can also be combined with other technologies, such as the discs, which can be sterilized and placed within 96-well plates. Moreover, it is amenable to other testing strategies, such as testing different antiseptic and disinfectant compounds.

The present disclosure demonstrates a methodology that allows simple, inexpensive, rapid and accurate testing of the in vitro susceptibility of microbial biofilms to antimicrobial agents. Because of the compatibility of the present disclosure with the 96-well microtiter platform and high throughput potential, the methods and devices disclosed herein should prove important in high-throughput susceptibility testing of biofilms, both as a research tool, and in the clinical and industrial laboratories. Use of this technology should be helpful for the selection of antimicrobial agents active against biofilms and for the screening of new effective antimicrobial agents (including antibiotics) to combat biofilm-associated infections and industrial contamination.

Disclosed is a multicompartment assay device comprising: at least one compartment comprising a viable organism control medium capable of sustaining growth of total microbial organisms; and, at least one compartment comprising an antimicrobial medium. The medium capable of sustaining growth of total microbial organisms can comprise a metabolic substrate comprising a detectable moiety capable of being released from the substrate by action of a microbial enzyme. The antimicrobial medium can comprise a metabolic substrate comprising a detectable moiety capable of being released from the substrate by action of a microbial enzyme. The medium capable of sustaining growth of total microbial organisms, and, the antimicrobial medium each may comprise an identical type of detectable signal. The antimicrobial medium can comprise any number of possible anti-microbial agents (e.g. amoxicillin, clavulanic acid/amoxicillin, enrofloxacin, cephalothin (cephalothin assay of often used to represent the efficacy of cephalothin, cephaprin, cephradine, cephalexin, cefaclor, and cefadroxil), gentamicin, and chloramphenicol).

Disclosed is a method of detecting the growth of microbial microorganisms in a biological sample and of simultaneously determining the susceptibility of such microorganisms to antimicrobial agents, the method comprising providing a multicompartment assay device comprising at least one compartment comprising a medium capable of sustaining growth of total microbial organisms in a biofilm, and, at least one compartment comprising an antimicrobial medium; placing a portion of the biological sample respectively in said at least one compartment comprising a medium capable of sustaining growth of total microbial organisms; and at least one compartment comprising an antimicrobial medium comprising an antimicrobial agent; whereby growth of organisms in the at least one compartment comprising a medium capable of sustaining growth of total microbial organisms on a support present in the at least one compartment indicates the presence of microbe in the sample; and growth of organisms in said at least one compartment comprising an antimicrobial medium indicates the affect of the antimicrobial agent on the microbes in a biofilm including growth and viability of the microbes.

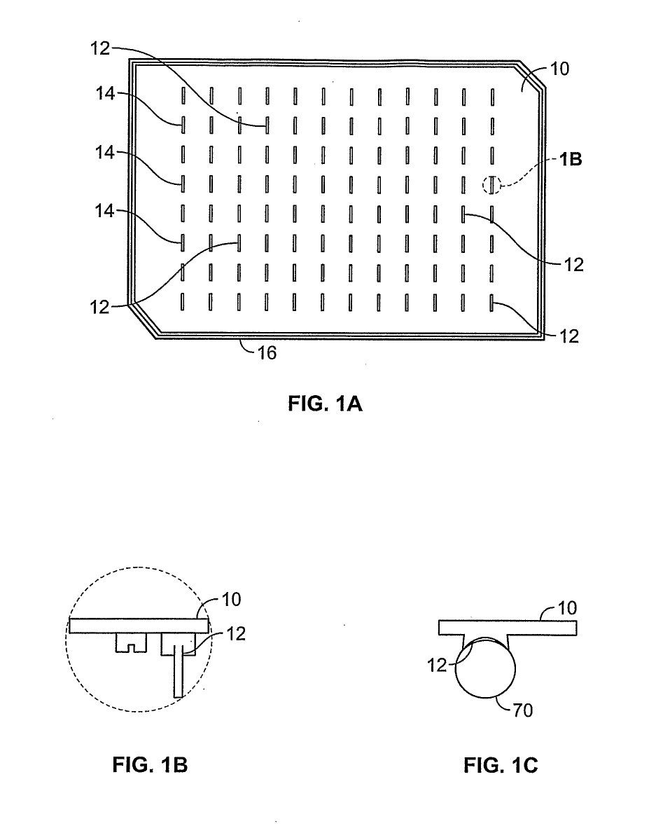

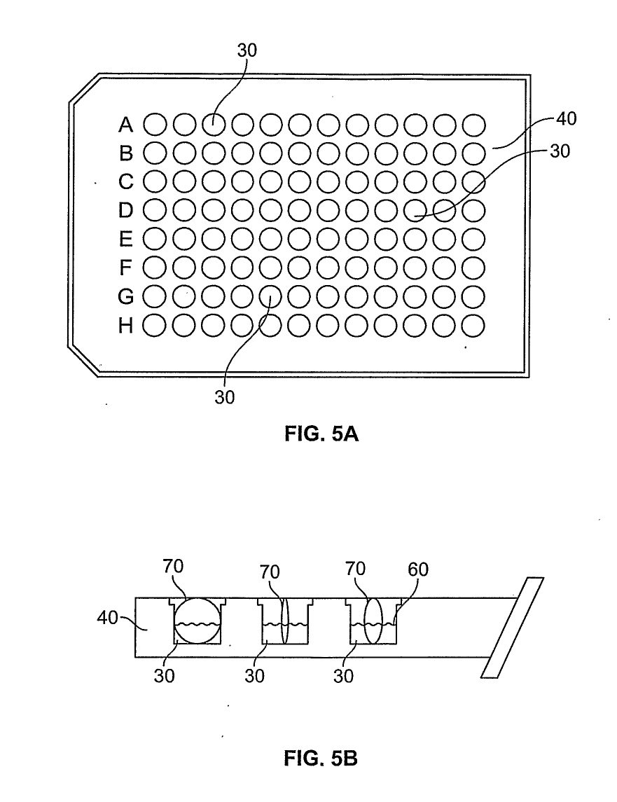

As shown in FIGS. 1, 2 and 3, a biofilm assay device includes a biofilm lid 10 composed of ELISA grade plastic or other suitable material (e.g. stainless steel, titanium). A plurality of projections 12 that fit within wells 30, or channels 24, of a microtiter plate. The projections 12 may be a support for biofilm attachment or may be attached to a support material for biofilm attachment. Accordingly, as used herein a projection comprises a support for biofilm adherent sites to which a biofilm may adhere. The projections 12 are designed to fit within a desired culture plate or multiwell plate. In one aspect, the projection 12 comprises a cleft which allows insertion of a disc or other support material. In certain embodiments a support such as, for example, an acetate disc is provided perpendicular to the bottom planar surface of a well or trough (see FIGS. 1B and 1C). In an alternative embodiment, discs equal to or slightly larger in diameter then the well may be inserted and held in place by friction. The number of projections 12 can be conveniently designed to match each well of a 96 well microtiter plate commonly used in biomedical assays. Each projection may be used to determine the initial biofilm concentration after incubation with or without antimicrobial agents. In another embodiment, the exemplary projections 12 are about 1.5 cm long and 2 mm wide.

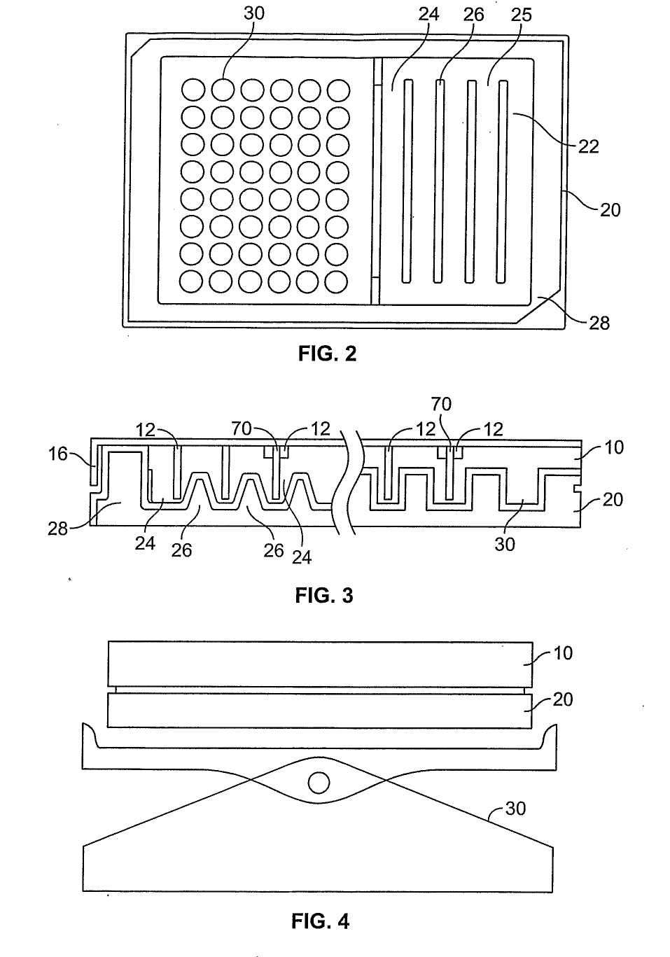

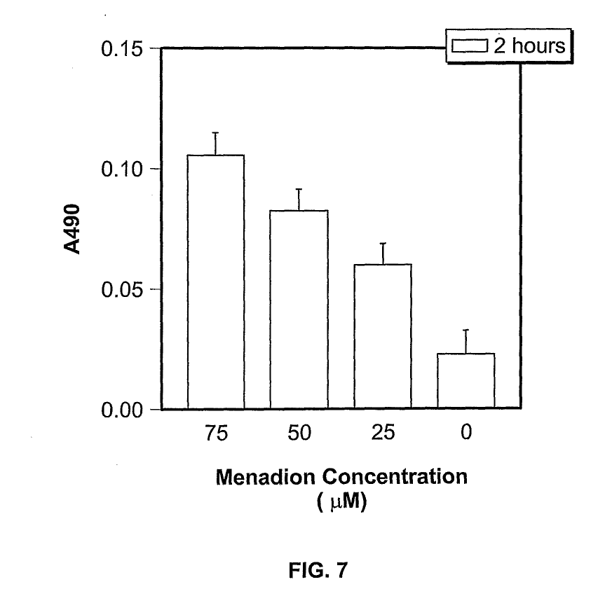

The biofilm assay device also includes a vessel 20. FIG. 2 depicts a vessel 20 having a liquid holding basin 22 divided into plural channels (troughs) 24 by molded ridges 26. The channels 24 are wide enough to receive the projections 12. There should be one channel 24 for each projection 12 of any given row 14. The lid 10 forms a foundation for the projections 12 for supporting the biofilm adherent sites within the channels 24. The lid 10 has a surrounding lip 16 that fits tightly over a surrounding wall 28 of the vessel 20 to avoid contamination of the inside of the vessel during incubation. Vessel 20 may also comprise a plurality of individual wells 30, each of which can be prepared to contain an antimicrobial agent. In another embodiment the vessel 20 comprises only a plurality of channels 24 and ridges 26. In another embodiment, the vessel 20 comprises only wells 30, such as the microtiter plate 40 depicted in FIG. 5.

The biofilm incubation vessel 20 serves two important functions for biofilm development. The first is as a reservoir for liquid growth medium containing the microbial population which will form a biofilm on the projections 12 or other support (e.g., discs 70) of the biofilm lid 10. The second function is to generate shear force across the projections or discs, which allows for optimal biofilm production on the projections or discs. In one aspect, the biofilms are grown with projections 12 or discs 70 located within channels 24 and wells 30. After a sufficient period of time and under appropriate conditions for biofilm growth, the lid 10 is removed and rotated 180 degrees such that the projections, supports or discs that were in the troughs 24 previously, are now located above wells 30. In this way, biofilm growth is effectively performed on the projections or discs originally located within troughs 24, upon rotating the lid 10 the projections, support or discs comprising biofilm are now located above the wells 30. The wells 30 may have previously contained various antimicrobial agents, or alternatively upon removing the lid 10, a desired one or more antimicrobial agents are added to the wells 30. The lid 10 is then replaced on vessel 20, such that the projections that were previously in troughs 24 are now in wells 30.

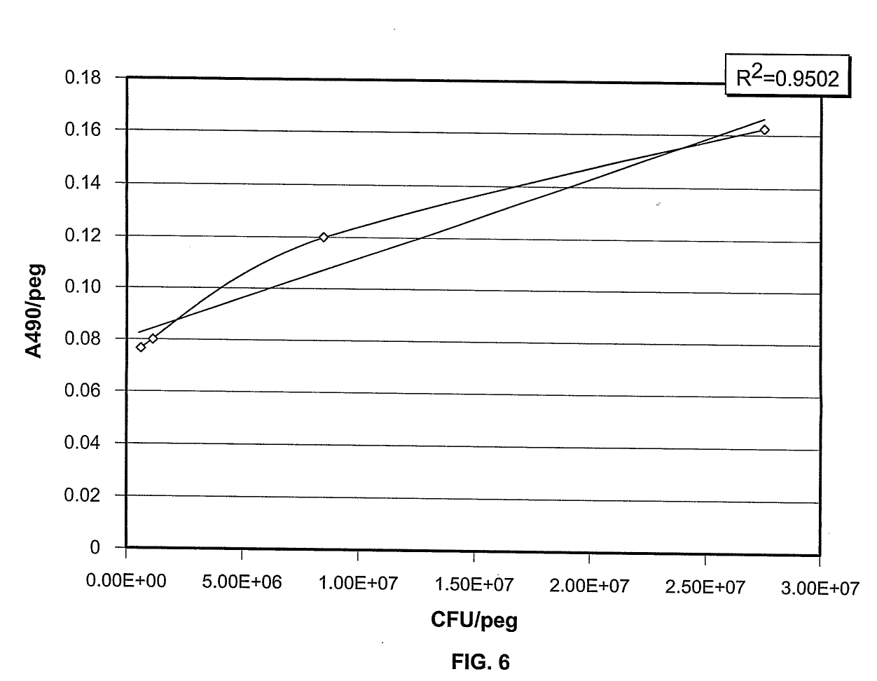

As shown, in FIG. 4, shear force on the projections 12 or discs 70 is generated by rocking the vessel 20 with lid 10 on a tilt table 30. The projections 12 or discs 70 sit suspended in the channels 24, or wells 30 so that the tips of the projections 12 or discs 70 may be immersed in liquid growth medium flowing in the channels 24 or in media 60 within wells 30. The ridges 26 channel the liquid growth medium along the channels 24 past and across the projections 12 or discs 70 suspended in the media, and thus generate a shear force across the projections or discs. Rocking of the vessel 10 causes a repeated change in direction of flow, in this case a repeated reversal of flow of liquid growth medium, across the projections 12 or discs 70, which helps to ensure a biofilm of equal proportion on each of the projections 12 or discs 70 of the lid 10. Rocking of the vessel, with liquid flowing backwards and forwards along the channels, provides an excellent biofilm growth environment that simulates natural occurring conditions of turbulent flow. Alternatively, when 96 well plates are used a gyrorotary shaker is used to generate a circulating flow.

Each projection 12 or disc 70, each channel 24, and each well 30 should have substantially the same shape to ensure uniformity of shear flow across the projections during biofilm formation. In addition, the uniform channels 24 should all be connected so that they share the same liquid nutrient and microbial mixture filling the basin 22. With sharing of the same microbial culture and channel configuration being the same for each channel, biofilms are produced at each projection or disc that are equivalent for the purpose of testing antimicrobial agents. In this way, different concentrations of different antimicrobials may be compared to each other without regard to positional variance of the projections during biofilm growth. Biofilms thus produced are considered to be uniform.

Sensitivity of a biofilm to antimicrobials or biocides, referred to in this disclosure collectively as “antimicrobial agent”, is measured by contacting the biofilm adherent sites (e.g., the biofilms grown on projections or discs) with an anti-microbial agent, and then assaying the biofilm. This may be accomplished by placing the lid 10 comprising projections 12 or discs 70, which were colonized with a biofilm in an incubation vessel 20, into a plurality of wells 30 on the opposite end of vessel 20, by rotating the lid 10 appropriately. Alternatively, the lid 10 comprising projections 12, or discs 70, comprising biofilms are placed into a conventional 96 well plate 40 such as illustrated in FIG. 5, the number of wells 30 being determined by the number of projections 12, in which growth medium containing an antimicrobial agent (e.g., an antibiotic or biocide) dilutions has been dispensed. The lid 10 and plate 40 fit such that microbial contamination from outside the plate cannot take place. Projections 12 or discs 70 that have been incubated in the same channel 24 or media of the vessel 20 should each be treated with a different antimicrobial agent. In this manner, consistent results may be obtained since the growth conditions in any one channel will be very similar along the entire channel and thus for each projection 12 or disc 70 suspended in that channel or a particular well. This helps improve the reliability of treatment of different projections 12 or discs 70 with different anti-microbial agents.

Each well 30 also comprises a signal generating substrate such as, for example, XTT that is used to determine the growth and viability of the biofilm in the presence and absence of various antimicrobial agents.

In alternative embodiments, specific test media that have been applied to the well series of the test device include general growth medium, cell-specific growth medium, and a series of antimicrobial media. The antimicrobial media test series may be selected from, but are not limited to, the tests for the antimicrobial efficacy of amoxicillin, enrofloxacin, clavulanic acid/amoxicillin, cephalothin (cephalothin assay of often used to represent the efficacy of cephalothin, cephaprin, cephradine, cephalexin, cefaclor, and cefadroxil (NCCLS Antimicrobial Susceptibility Testing/SC3, January, 1996)), gentamicin, and chloramphenicol, and the like. Furthermore, wells comprising an antimicrobial agent may comprise a series of dilutions of the same antimicrobial agent to determine the lethal dose or inhibitory concentration of the antimicrobial agent.

In another aspect, samples from a device or site of infection in vivo are obtained to determine an effective antibacterial therapy. The method uses microbial culture media which allows detecting the pathogens, and also uses antimicrobial media for determining the antimicrobial efficacy of selected antimicrobial agents towards the detected pathogens. For example, a specimen obtained from a patient suspected of having an infection is added to a series of wells comprising a projection or disc, microbiological growth media containing one or more hydrolyzable fluorogenic or colorigenic substrates (e.g. XTT); the series of growth media include general growth medium, pathogen/cell-specific growth medium, and antimicrobial media series. These test materials and processes can, in certain cases also be arranged to allow conventional microbiological culture to be continued so that the exact identity of a pathogen and the quantitative antimicrobial susceptibility information obtained later as a confirmation if desired.

Although the invention has been generally described above, further aspects of the invention will be apparent from the specific disclosure that follows, which is exemplary and not limiting.

### EXAMPLES

Biofilm Growth Conditions. Isolates were propagated in Mueller-Hinton broth (MHB). Flasks containing liquid medium (20 ml) were inoculated with a loopful of cells from MHB agar plates containing freshly grown isolates, and incubated overnight in an orbital shaker (100 rpm) at 37° C. Cells were harvested and resuspended in MHB at a cellular density equivalent to 1.0×10⁶ cells per milliliter. Biofilms were formed on the pegs of the Calgary Biofilm Device (CBD; MBE™ Biofilm Technologies, Ltd., Edmonton AB), described in Ceri et al. (Ceri et al., J. Clin. Microbiol., 37(6):1771-6, 1999). The device consists of 96 conical pegs attached to a plastic lid. Biofilms were formed on the CBD by placing the device lid in a 96-well microtiter plate containing 200 μl of bacterial inoculum. The device was placed on a rocking platform at 37° C. and 95% humidity for selected time intervals (depending upon experiment), after which the lid was removed and briefly rinsed in phosphate buffered saline (PBS) to remove loose biomass.

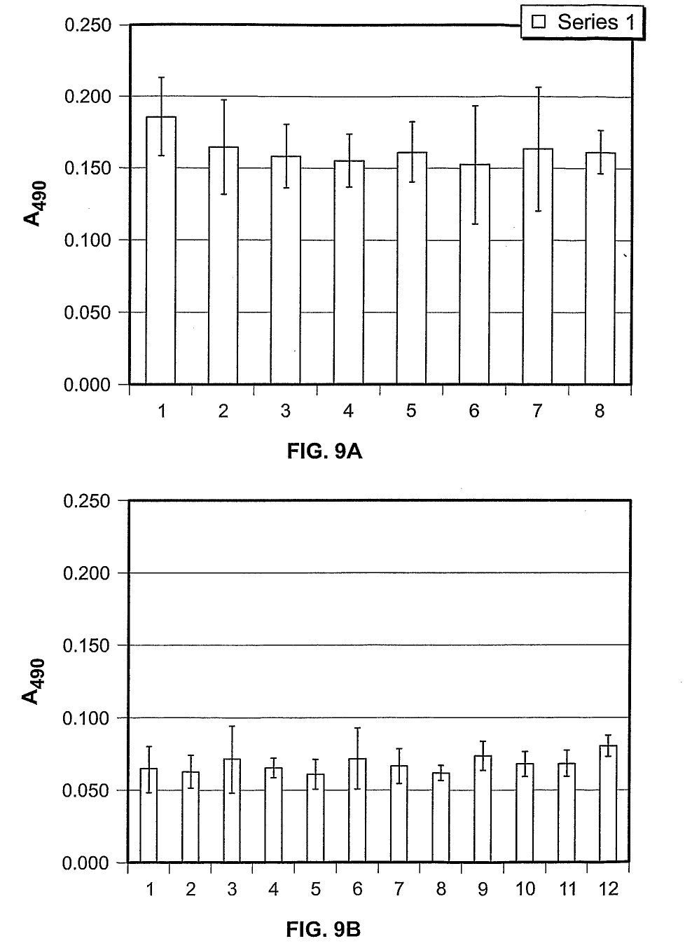

Optimizing XTT Assay. In order to optimize the quantities of 2,3-bis[2-methyloxy-4-nitro-5-sulfophenyl]-2H-tetrazolium-5-carboxanilide (XTT; Sigma) and menadione required for quantifying biofilm metabolic activity, varying concentrations were tested. P. aeruginosa PAO1 was used for these experiments. XTT was dissolved in sterile phosphate buffered saline. It was previously demonstrated that a saturated solution of 0.5 mg/ml of XTT was optimal (Ramage et al. 2001). The results obtained were in agreement with the prior observations. The concentration of menadione (2-methyl-1,4-naphthoquinone; Sigma), the electron-coupling agent, was varied to establish the optimal molarity in respect to maximal absorbance readings. The varying concentrations (0, 25, 50 and 75 μM) were dissolved in 100% acetone and stored at −70° C. until required. Preformed biofilms were immersed in 200 μl of the varying concentrations of menadione. The change in XTT absorbance at 490 nm was assessed spectrophotometerically using an automated microtiter plate reader. It was determined that 75 μM gave the optimal change in absorbance after 2 h (FIG. 7). XTT at 0.5 mg/ml and menadione at 75 μM concentrations were used throughout the following experiments.

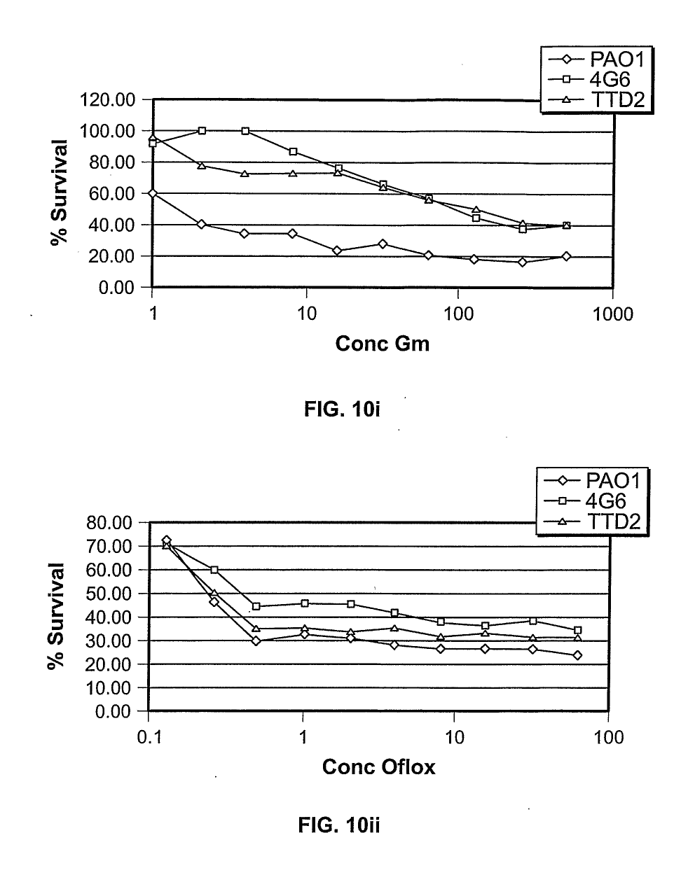

Comparison of XTT Absorbance and Colony Forming Units. The validity of the assay was tested with respect to the change in absorbance in relation to an increase in viable bacterial counts within biofilms, to establish whether there was a direct correlation. P. aeruginosa PAO1 was used for these experiments. Biofilms were formed on the CBD over 72 h (4, 6, 8, 10, 24, 48 and 72 h) and measured throughout in terms of XTT absorbance. Cfu/ml was determined by standard plate counting methodology. The absorbance change was measured after 2 hours. An increase in XTT absorbance was related to an increase in cfu/ml on the peg of the CBD. Biofilm growth curves are illustrated in FIG. 8. It was demonstrated that a minimum density of 10⁵ cfu/ml was required to elicit a change in XTT absorbance. Between the densities of 1×10⁵ to 3×10⁷ cfu/ml there was a good correlation between cfu/ml and change of XTT absorbance (R²=0.9502) (FIG. 6). Bacterial densities greater than 3×10⁷ were not achieved for biofilm mode of growth upon the peg.

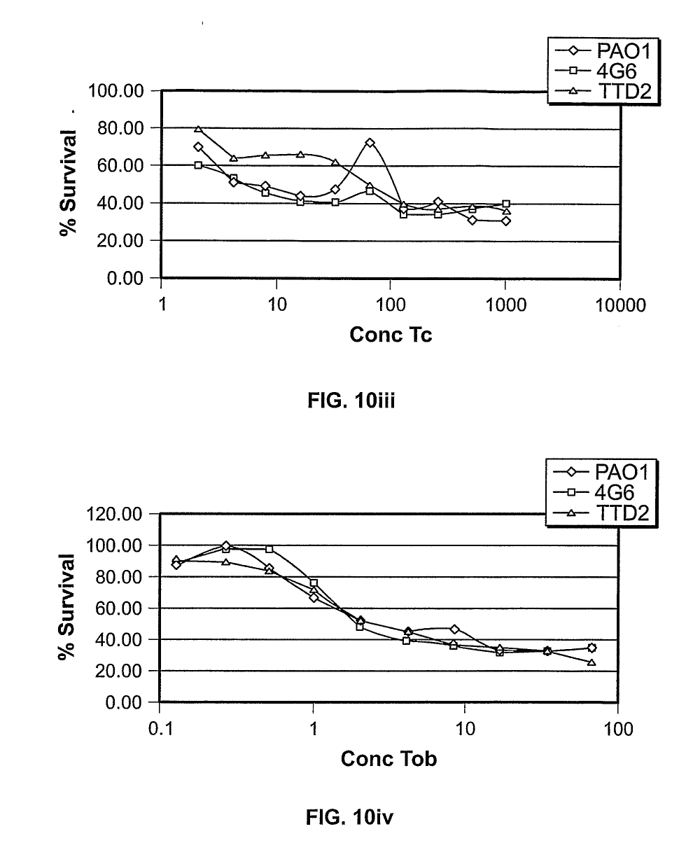

Formation of Equivalent Biofilms. Biofilms were formed on the CBD as described above, and measured after 48 h with the optimized metabolic assay. Biofilms were compared both along the rows and the columns of the CBD. It was demonstrated that there was no significant difference between the biofilms formed along the rows and the columns of the CBD, as assessed by XTT absorbance (FIGS. 9A and 9B).

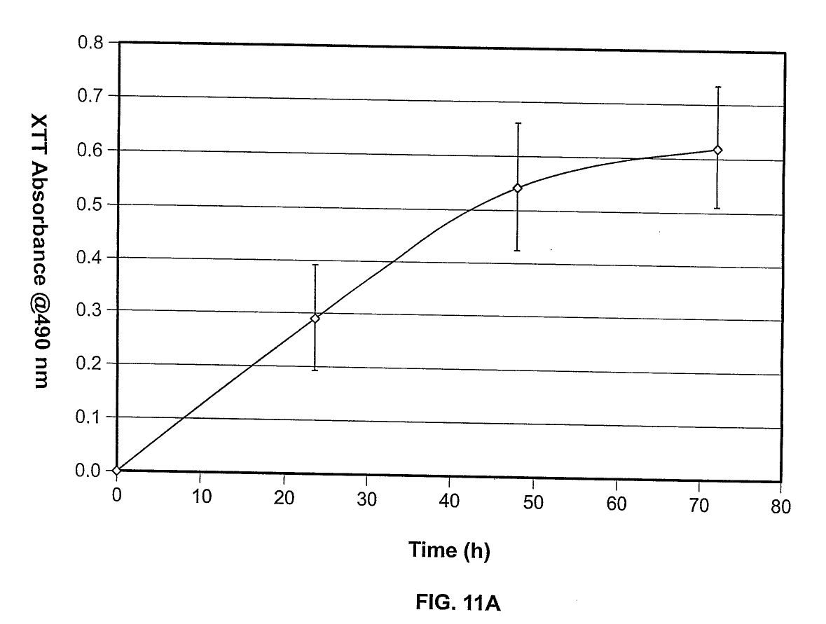

Assessment of Biofilm Killing Kinetics Using XTT. Biofilms of P. aeruginosa PAO1, and two mutant derivative strains 4G6 and TTD2, were formed on the CBD for 48 h. Following biofilm formation, the medium was aspirated and non-adherent cells removed by gently washing the biofilms three times in sterile PBS. Antibiotics (gentamicin, ofloxacin, tetracycline and tobramycin) were serially double diluted (1024 to 1 μg/ml) in MHB along the rows of a Nunc 96-well microtitre plate. Biofilms were then immersed into the antibiotic solutions and incubated for 6 h at 37° C. A series of antibiotic free wells and biofilm-free wells were also included to serve as positive and negative controls, respectively. XTT absorbance readings were taken at 2 h and then at 24 h. Killing of the biofilm was assessed as percent reduction in absorbance as compared to the unchallenged control biofilms. It was shown that a dose dependent effect was observed for all strains and all antibiotics (FIG. 10i, ii, iii, iv). Strains 4G6 and TTD2 were less susceptible to gentamicin, as assessed by XTT absorbance. All biofilms were shown to be resistant to the antibiotics, i.e. 99.9% of the biofilm were not killed following antibiotic challenge. Residual metabolic activity of the biofilms demonstrated inefficient pharmacokinetics.

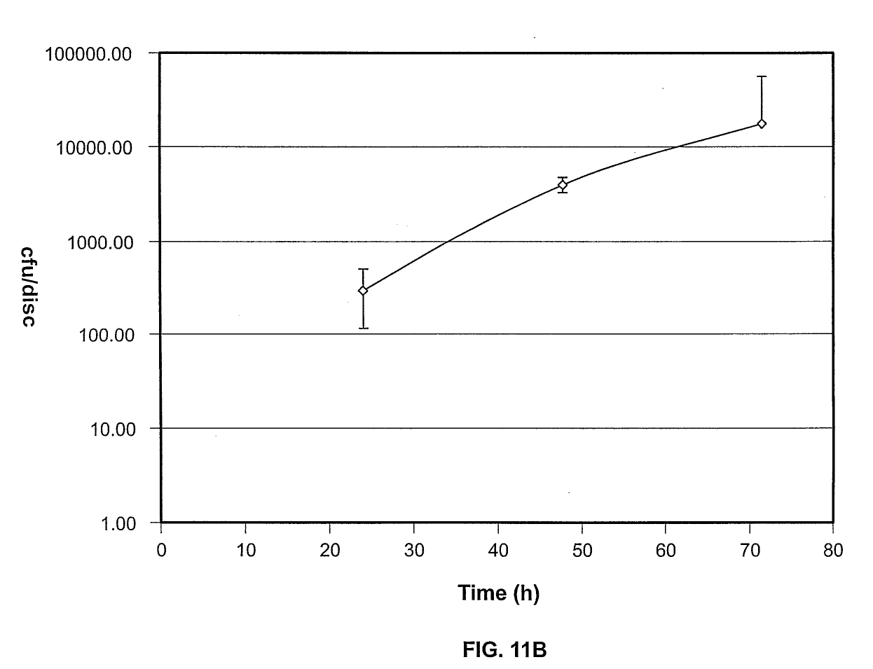

Discs. Acetate discs (approximately 5 mm in diameter) were cut from commercially available overhead transparencies. The acetate discs were soaked in 70% ethanol, and then subsequently sterilized by ultraviolet light exposure overnight. Discs were placed inside wells of a 96-well microtitre plate, perpendicular to the wells to provide an air/liquid interface optimal for biofilm formation. Two hundred μl of P. aeruginosa PA01 inoculum was then added to each well. The plate was placed on a rocking platform at 37° C. and 95% humidity for 24, 48 and 72 h. Biofilms were then removed, washed gently in PBS, immersed in 150 μl of XTT and incubated at 37° C. for 3 h. In parallel, viable cell counting was performed. It was demonstrated that for both XTT absorbance readings and cfu/ml, a similar growth curve pattern was observed (FIGS. 11A and B, respectively).

Based upon the foregoing, preformed biofilms on the pins or multiwell plates were shown to undergo a dose dependent killing as determined by XTT absorbance. Moreover, in the P. aeruginosa mutant strains 4G6 and TD2, which contained a gentamicin cassette, decreased susceptibility was observed, i.e. an increased XTT absorbance reading was detected.

It must be noted that as used herein and in the appended claims, the singular forms “a,” “and,” and “the” include plural referents unless the context clearly dictates otherwise. Thus, for example, reference to “a formulation” includes mixtures of different formulations and reference to “the method of treatment” includes reference to equivalent steps and methods known to those skilled in the art, and so forth.

Unless defined otherwise, all technical and scientific terms used herein have the same meaning as commonly understood by one of ordinary skill in the art to which this invention belongs. Although any methods and materials similar to equivalent to those described herein can be used in the practice or testing of the invention, the preferred methods and materials are now described. All publications and documents mentioned herein are fully incorporated by reference.

A number of embodiments of the invention have been described. Nevertheless, it will be understood that various modifications may be made without departing from the spirit and scope of the invention. Accordingly, other embodiments are within the scope of the following claims.

What is claimed is:

1. A method of determining the susceptibility of a biofilm to an antimicrobial agent, comprising:
culturing microbes on a support to form a biofilm;
contacting the biofilm with a metabolic substrate;
determining a base-line metabolic activity of the biofilm by measuring a signal from the metabolic substrate;
contacting the biofilm with one or more antimicrobial agents;
determining an experimental metabolic activity by measuring a signal from the metabolic substrate; and
comparing the base-line metabolic activity with the experimental metabolic activity, wherein a change is indicative of an antimicrobial agent that affects microbes in the biofilm.

2. The method of claim 1, wherein the metabolic substrate comprises a fluorogenic or chromogenic moiety.

3. The method of claim 1, wherein the metabolic substrate comprises a member selected from the group consisting of nitroblue tetrazolium chloride BT; 2H-(Tetrazolium,-3,3′-(3,3′-dimethoxy(1,1′-biphenyl)-4,4′-diyl)bis(4-nitro phenyl)-5-(phenyl), dichloride); 3-(4,5-Dimethyl-2-thiazolyl)-2,5-diphenyl-2H-tetrazolium bromide (MTT; thiazolyl blue); 2-(p-iodophenyl)-3-(p-nitrophenyl)-5-phenyltetrazolium chloride (INT); 3-(4-Iodophenyl)-2-(4-nitrophenyl)-5-phenyl-2H-tetrazolium chloride; neotetrazolium chloride (NTC; 2,2′,5,5′-Tetraphenyl-3,3′-[p-diphenylene] ditetrazolium chloride); tetranitro tetrazolium blue chloride (TNBT; 2,2′,5,5′-Tetra(4-nitrophenyl)-3,3′-dimethoxy-4,4′-biphenylene)-2H,2H′-ditetrazolium chloride); tetrazolium Blue chloride (BT; blue tetrazolium chloride; 2,2′,5,5′-Tetraphenyl-3,3′-(3,3′-dimethoxy-4,4′-biphenylene)-2H,2H′-ditetrazolium chloride); triphenyltetrazolium chloride (TTC; tetrazolium red; 2,3,5-Triphenyl-2H-tetrazolium chloride); triphenyltetrazolium bromide (TTB; 2,3,5-Triphenyl-2H-tetrazolium bromide); 4-[3-(4-Iodophenyl)-2-(4-nitrophenyl)-2H-5-tetrazolio]-1,3-benzene disulfonate (WST 1); 4-[3-(4-Iodophenyl)-2-(2,4-dinitrophenyl)-2H-5-tetrazolio]-1,3-benzenedisulfonate (WST 3); 2-Benzothiazolyl-3-(4-carboxy-2-methoxyphenyl)-5-[4-(2-sulfoethylcarbamoyl)phenyl]-2H-tetrazolium salt (WST 4); 2,2′-dibenzothiazolyl-5,5′-bis(4-di(2-sulfoethyl)carbamoylphenyl)-3,3′-(3,3′-dimethoxy-4,4′-biphenylene)ditetrazolium, disodium salt (WST-5); sodium 3′-{1-[(phenylamino)-carbonyl]-3,2,3-bis[2-methyloxy-4-nitro-5-sulfophenyl]-2H-tetrazolium-5-carboxanilide (XTT); 2-(2′-benzothiazolyl)-5-styryl-3-(4′-phthalhydrazidyl) tetrazolium (BSPT); 2-benzothiazolyl-(2)-3,5-diphenyl tetrazolium (BTDP); 2,3-di(4-nitrophenyl)tetrazolium (DNP); 2,5-diphenyl-3-(4-styrylphenyl) tetrazolium (DPSP); distyryl nitroblue tetrazolium (DS-NBT); 2-phenyl-3-(4-carboxyphenyl)-5-methyl tetrazolium (PCPM); thiocarbamyl nitroblue tetrazolium (TCNBT; 2,2′-Di(p-nitrophenyl)-5,5′-di(p-thiocarbamylphenyl)-3,3′-(3,3′-dimethoxy-4,4′-biphenylene)ditetrazolium chloride); 5-cyano-2,3-di-4-tolyl-tetrazolium chloride (CTC); Nitrotetrazolium Violet (NTV); p-Anisyl Blue Tetrazolium Chloride (pABT); m-Nitro Neotetrazolium Chloride (m-NNT); o-Tolyl Tetrazolium Red (o-TTR); p-Tolyl Tetrazolium Red (pTTR); Piperonyl Tetrazolium Blue (PTB); p-Anisyl-p-Nitro Blue Tetrazolium Chloride (pApNBT); Veratryl Tetrazolium Blue (VTB); and tetrazolium violet (TV; 2,5-Diphenyl-3-(alpha-naphthyl)tetrazolium chloride).

4. The method of claim 1, wherein the support is a Calgary Biofilm Device.

5. The method of claim 1, wherein the support comprises discs in a culture well.

6. The method of claim 5, wherein the discs comprise an acetate material.

7. The method of claim 1, further comprising one or more additional metabolic substrates.

8. An assay device comprising:
a cell culture device comprising a plurality of wells, each well comprising a substantially planar bottom and at least one wall;
a plurality of supports, each support disposed within a well perpendicular to the substantially planar bottom, wherein the plurality of supports comprise discs; and
at least one cover that fittably seals the top of each well.

9. The assay device of claim 8, wherein the cell culture device comprises a 96-well tissue culture plate.

10. The assay device of claim 8, wherein the plurality of supports are acetate discs.

11. The assay device of claim 8, wherein each of the plurality of discs is treated with ethanol and washed prior to being disposed within each well.

12. The assay device of claim 8, wherein the diameter of the discs is equal to, or slightly larger than the diameter of each well.

13. An assay system comprising:
a cell culture device comprising a plurality of wells and/or channels, each well or channel comprising a substantially planar bottom;
a plurality of supports, each support disposed within a well or channel perpendicular to the substantially planar bottom; and
at least one cover that fittably seals the top of each well or channel;
culturing a sample comprising a microbial population in a media within the wells or channels such that the media is in contact with the supports thereby forming a biofilm on the supports;
measuring a fluorometric or calorimetric absorbance from a fluorogenic or chromogenic moiety in the sample;
comparing the fluorogenic or calorimetric absorbance to a standard sample.

14. The assay system of claim 13, wherein the cell culture device comprises a 96-well tissue culture plate.

15. The assay system of claim 13, wherein the plurality of supports comprise discs.

16. The assay system of claim 15, wherein the plurality of supports are acetate discs.

17. The assay system of claim 16, wherein each of the plurality of acetate discs is treated with ethanol and washed prior to being disposed within each well.

18. The assay system of claim 15, wherein the diameter of the discs is equal to, or slightly larger than the diameter of each well.

19. The assay system of claim 13, wherein the sample is an environmental sample.

20. The assay system of claim 13, wherein the sample is obtained from a patient.

21. The assay system of claim 13, wherein the microbial population is substantially homogeneous.

22. The assay system of claim 13, wherein the microbial population comprises a mixed species biofilm.

23. The assay system of claim 22, wherein the mixed species biofilm comprises a population of prokaryotes and eukaryotes.

24. The assay system of claim 23, wherein the eukaryotes comprise fungi or yeast microbes.

25. The assay system of claim 13, wherein the fluorogenic or chromogenic substrate is 2,3-bis[2-methyloxy-4-nitro-5-sulfophenyl]-2H-tetrazolium-5-carboxanilide (XTT).

26. The assay system of claim 13, wherein the support is affixed to the at least one cover.

27. The assay system of claim 25, further comprising:
means for measuring a base-line metabolic activity of the biofilm;
means for contacting the biofilm with one or more antimicrobial agents;
means for measuring an experimental metabolic activity; and
means for comparing the base-line metabolic activity with the experimental metabolic activity, wherein a change is indicative of an antimicrobial agent that affects microbes in the biofilm.
